(19) United States
(12) Patent Application Publication
(10) Pub. No.: US 2015/0374552 A1
(43) Pub. Date: Dec. 31, 2015

(54) Bandage for absorbing sweat produced by the toes

(71) Applicant: Chien Chung Lee, New Taipei City (TW)

(72) Inventor: Chien Chung Lee, New Taipei City (TW)

(21) Appl. No.: 14/852,642

(22) Filed: Sep. 14, 2015

(51) Int. Cl. A61F 13/00

(52) U.S. Cl. CPC A61F 13/00042; A61F 13/00029; A61F 13/00038; A61F 13/00046

(57) ABSTRACT

In one embodiment a bandage worn on the toes for absorbing perspiration includes two elastic backing layers each having a plurality of holes, first ends of the elastic backing layers being secured together, and second ends of the elastic backing layers being secured together; an absorbent pad placed between the elastic backing layers and attached thereto; and an adhesive strip having two ends adhesively attached to the first and second ends of one elastic backing layer respectively. The absorbent pad communicates with the holes.

### BACKGROUND OF THE INVENTION

1. Field of the Invention

The invention relates to personal hygienic items and more particularly to a bandage worn on the toes for absorbing perspiration.

2. Description of Related Art

One of the roles of socks is absorbing perspiration. The foot is a heavy producer of sweat in the body. Socks help to absorb this sweat and draw it to areas where air can evaporate the perspiration.

Tinea pedis is caused by parasitic fungi. These parasitic fungi are typically transmitted in moist communal areas and require a warm moist environment like the inside of a shoe to incubate. Fungal infection of the foot may be acquired by wearing fungi-contaminated socks or shoes. Toe nails may become infected with fungi in the same way as the rest of the foot, typically by being trapped with fungi in the warm, dark, moisture inside of a shoe.

The infection can be prevented by keeping the feet dry. However, as a means for preventing the parasitic fungi from transmitting to the toes, a bandage worn on the toes for absorbing perspiration has not been disclosed as far as the present inventor is aware.

### SUMMARY OF THE INVENTION

It is therefore one object of the invention to provide a bandage comprising two elastic backing layers each having a plurality of holes, first ends of the elastic backing layers being secured together, and second ends of the elastic backing layers being secured together; an absorbent pad placed between the elastic backing layers and attached thereto; and an adhesive strip having two ends adhesively attached to the first and second ends of one elastic backing layer respectively; wherein the absorbent pad communicates with the holes.

The above and other objects, features and advantages of the invention will become apparent from the following detailed description taken with the accompanying drawings.

### BRIEF DESCRIPTION OF THE DRAWINGS

FIG. 1 is an exploded view of a bandage according to a first preferred embodiment of the invention;

FIG. 2 is a sectional view of the formed bandage;

FIG. 3 is an environmental view of the bandage worn on the toes but not fastened;

FIG. 4 is a view similar to FIG. 3 showing the fastened bandage;

FIG. 5 is a schematic front view of FIG. 4;

FIG. 6 is an environmental view of a bandage according to a second preferred embodiment of the invention, the bandage being worn on the toes but not fastened;

FIG. 7 is a view similar to FIG. 6 showing the fastened bandage; and

FIG. 8 is a schematic front view of FIG. 7.

### DETAILED DESCRIPTION OF THE INVENTION

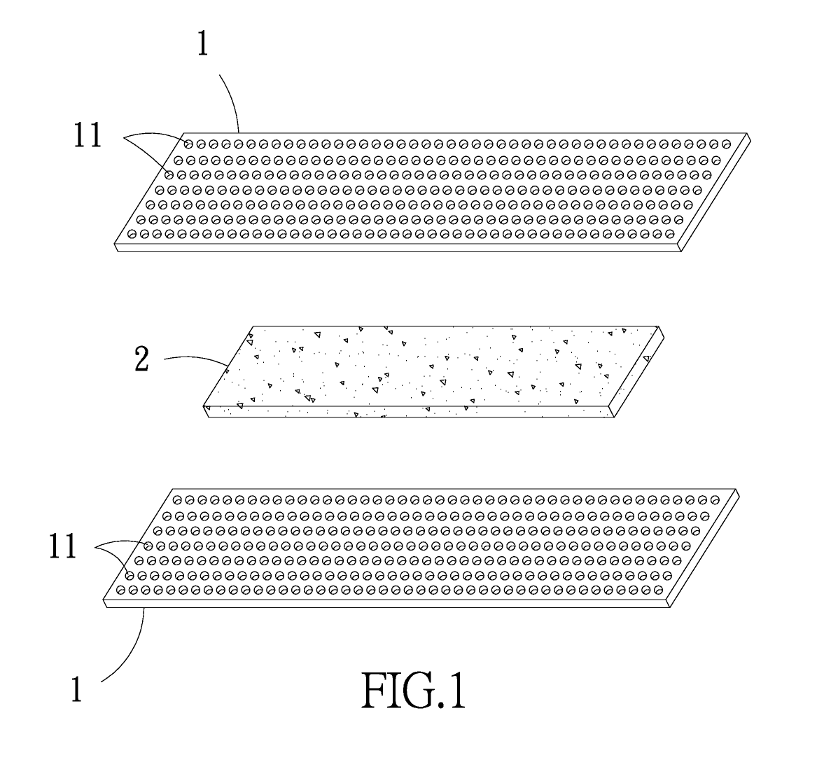

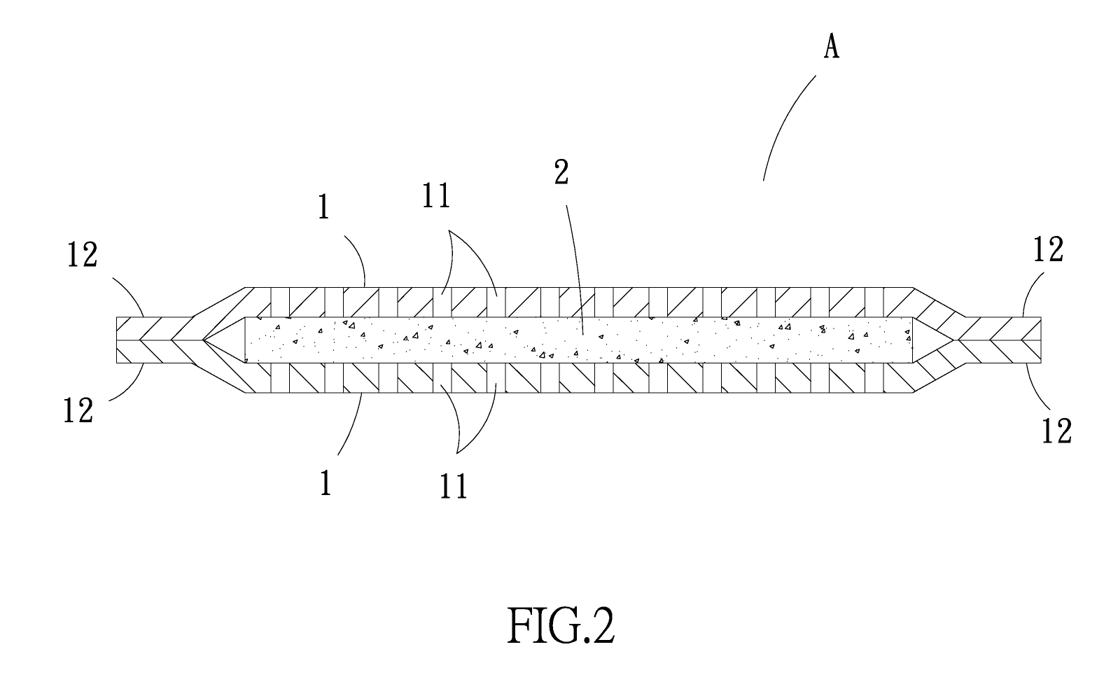

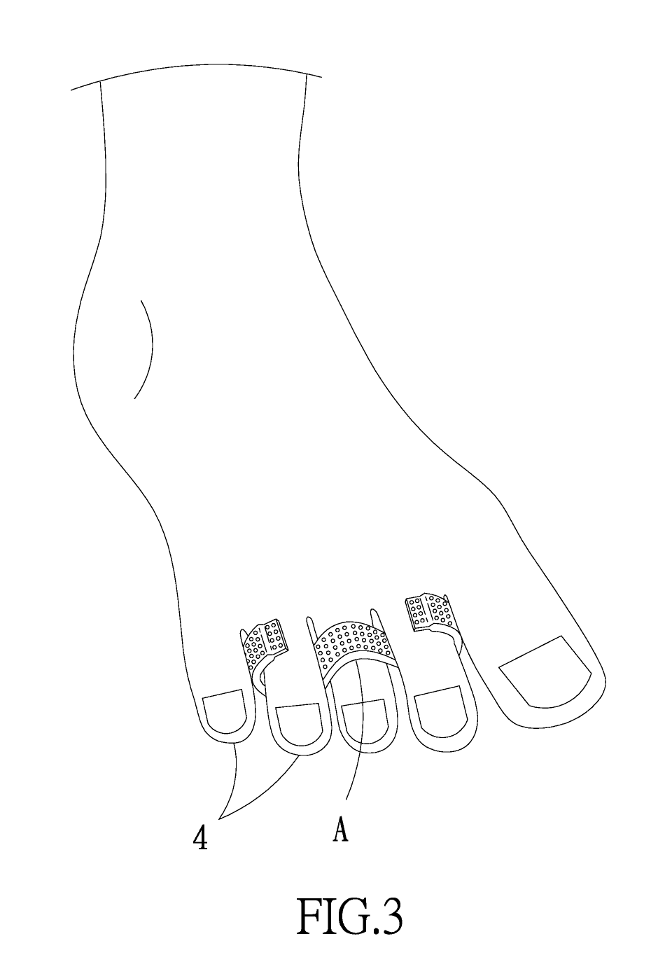

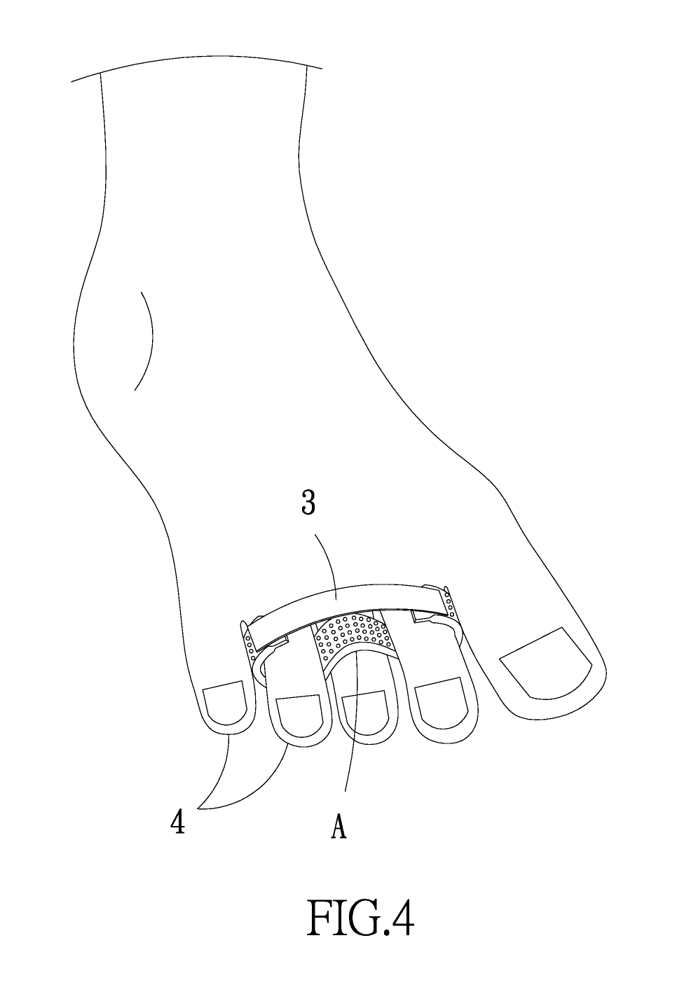

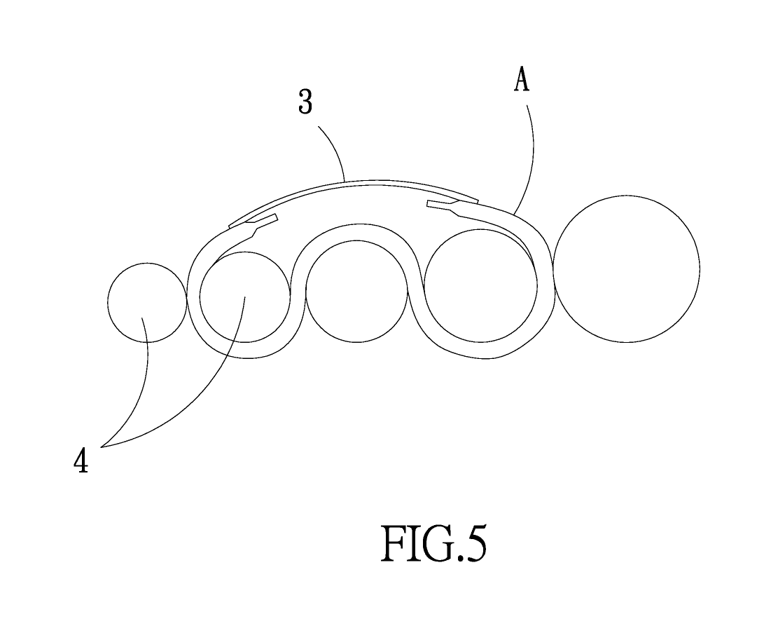

Referring to FIGS. 1 to 5, a bandage A in accordance with a first preferred embodiment of the invention comprises the following components as discussed in detail below.

Two elastic backing layers 1 are rectangular and each has a plurality of holes 11. An absorbent pad 2 is placed between the elastic backing layers 1 and attached thereto. A rectangular adhesive strip 3 has two ends adhesively attached to two adhesively attached ends 12 of the elastic backing layers 1 respectively for fastening the bandage A worn on the toes 4. As such, the absorbent pad 2 can absorb sweat produced by the toes 4 through the holes 11.

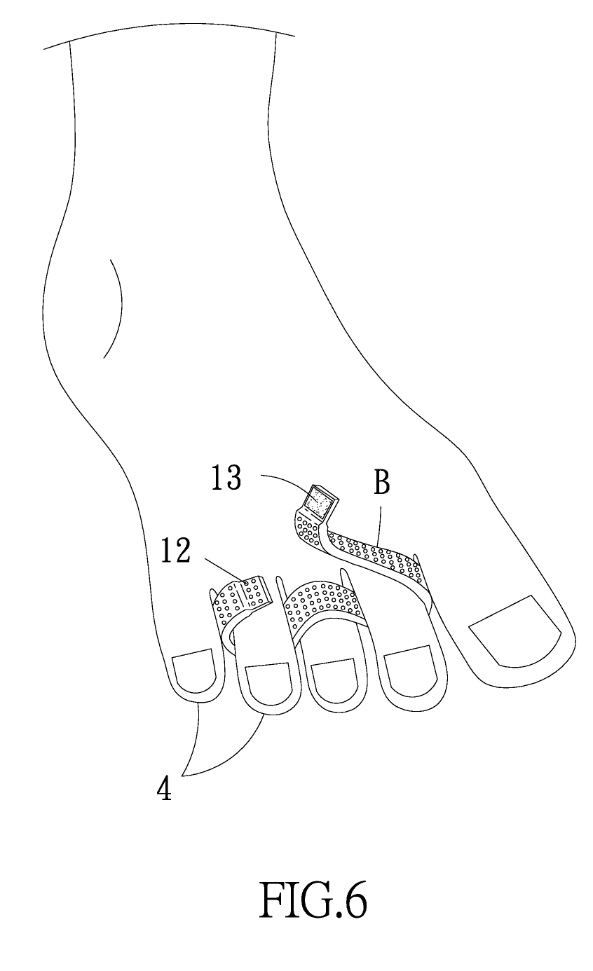

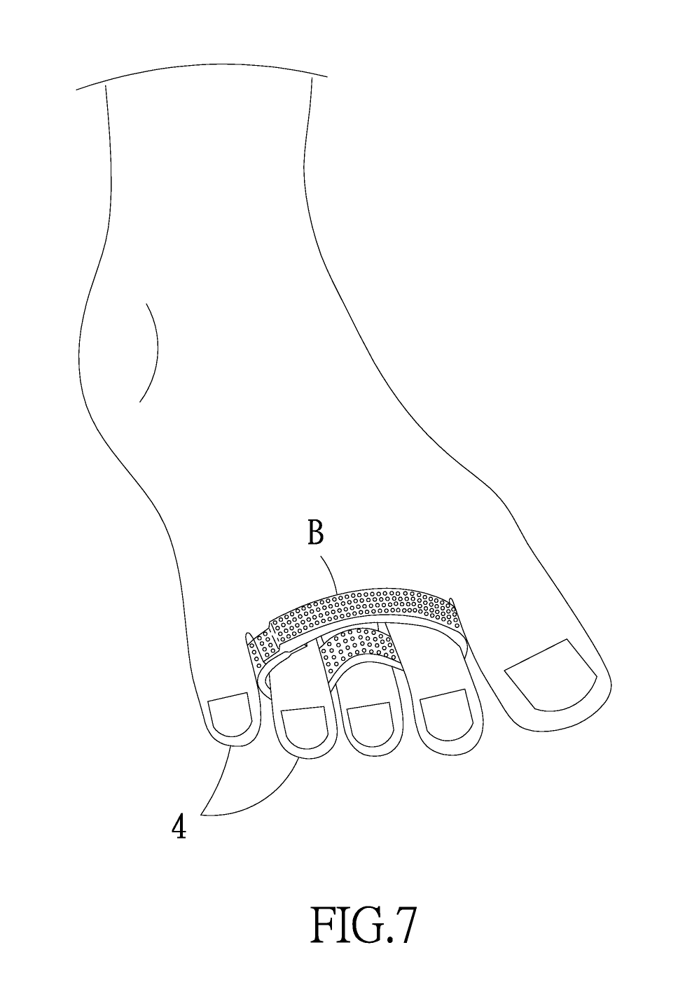

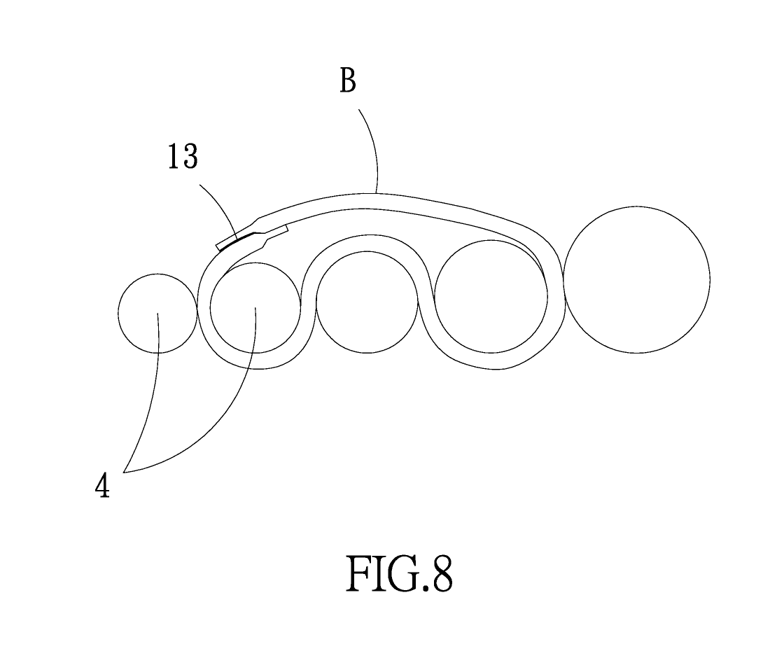

Referring to FIGS. 6 to 8, a bandage B in accordance with a second preferred embodiment of the invention is shown. The characteristics of the second preferred embodiment are substantially the same as that of the first preferred embodiment except the following: An adhesive member 13 is formed on a bottom of one end 12 of one of the elastic backing layers 1 and configured to adhesively attached to the other end 12 of the other elastic backing layer 1 for fastening the bandage B worn on the toes 4.

While the invention has been described in terms of preferred embodiments, those skilled in the art will recognize that the invention can be practiced with modifications within the spirit and scope of the appended claims.

What is claimed is:

1. A bandage comprising:
two elastic backing layers each including a plurality of holes, first ends of the elastic backing layers being secured together, and second ends of the elastic backing layers being secured together;
an absorbent pad placed between the elastic backing layers and attached thereto; and
an adhesive strip having two ends adhesively attached to the first and second ends of one elastic backing layer respectively;
wherein the absorbent pad communicates with the holes.

2. A bandage comprising:
two elastic backing layers each including a plurality of holes, first ends of the elastic backing layers being secured together, and second ends of the elastic backing layers being secured together;
an absorbent pad placed between the elastic backing layers and attached thereto; and
an adhesive member formed on a bottom of the first end of one elastic backing layer and configured to adhesively attach to the second end of the other elastic backing layer;
wherein the absorbent pad communicates with the holes.
